(19) United States
(12) Patent Application Publication
(10) Pub. No.: US 2022/0087742 A1
(43) Pub. Date: Mar. 24, 2022

(54) ABLATION CATHETERS AND SYSTEMS

(71) Applicant: Oscor Inc., Palm Harbor, FL (US)

(72) Inventors: Noel Perez, Palm Harbor, FL (US); Thomas P. Osypka, Palm Harbor, FL (US)

(73) Assignee: Oscor Inc., Palm Harbor, FL (US)

(21) Appl. No.: 17/482,212

(22) Filed: Sep. 22, 2021

Related U.S. Application Data
(60) Provisional application No. 63/082,398, filed on Sep. 23, 2020.

(51) Int. Cl. A61B 18/14

(52) U.S. Cl. CPC A61B 18/1492; A61B 2018/0016

(57) ABSTRACT

A catheter assembly can include an elongated catheter having opposed proximal and distal end portions and having an axial lumen extending therethrough, the axial lumen configured to accommodate electrical wires. The assembly can include a distal electrode array configured to have a basket shape in a deployed position and configured to collapse in a collapsed position, the distal electrode array configured to extend from the distal end portion of the elongated catheter in the deployed position and to be selectively contained within the axial lumen or other sheath in the collapsed position.

### CROSS-REFERENCE TO RELATED APPLICATIONS

This application claims priority to and the benefit of U.S. Provisional Application No. 63/082,398, filed Sep. 23, 2020, the entire contents of which are herein incorporated by reference in their entirety.

### FIELD

This disclosure relates to ablation catheters and systems, e.g., to ablation catheters for performing renal denervation procedures through the renal artery of a patient.

### BACKGROUND

Renal denervation (RDN) is a procedure performed by interventional radiologists for the purpose of lowering the blood pressure of a patient. Renal denervation is a minimally invasive, endovascular catheter-based procedure using radiofrequency (RF) ablation aimed at treating resistant hypertension.

By applying RF energy to the renal arteries, the nerves in the vascular wall (adventitia layer) can be denervated. This causes reduction of renal sympathetic afferent and efferent activity which in turn can decrease blood pressure. Early data from international clinical trials demonstrates average blood pressure reduction of approximately 30 mm Hg at three-year follow-ups in patients with treatment-resistant hypertension.

A common way to perform renal ablation is to ablate the renal artery by either heating tissue through radiofrequency or microwave ablation, irrigated heat ablation, and/or cryogenic ablation. It is believed that renal denervation works because it reduces the over-activity of the sympathetic nerve. Ablation of the renal artery is commonly performed by gaining access through the femoral vein. However, in certain cases, this can cause substantial bleeding. Other options include access through the radial artery. But this method limits the use of catheter systems of SF (French size) or smaller.

Current ablation catheters that are available to the market include: 1) single polar catheters offered by Medtronic of 45 710 Medtronic Parkway, Minneapolis, Minn., 55432-5604, which take substantial time to perform effective ablation of the renal artery; 2) cage form catheters offered by St. Jude Medical of One St. Jude Medical Drive, St. Paul, Minn., 55117-9983, which have several electrodes configured in a cage form; and 3) multiple ablation electrodes configured on an inflatable balloon, like those offered by Boston Scientific of One Boston Scientific Place, Natick, Mass., 01760-1537.

All unipolar renal denervation catheters have a major disadvantage. Unipolar catheters requires the use of a monopolar-based patient grounding pad therefore the ablation energy applied through the positive electrode or electrodes flows throughout the body into the grounding pad. Current will flow between the unipolar electrode or electrodes from inside the renal artery to the grounding pad typically located on the patient back. Current flow between the two electrodes will increase the temperature of the tissue and organs located between the two electrodes and physician cannot control the tissues or organs impacted by the temperature increase. The unipolar catheter also requires higher treatment time to achieve the RF ablation due to the grounding pad location. Additionally, unipolar RDN catheters require the use of cooling and irrigation systems to prevent overheating and damaging the renal artery walls and the circulating blood during the RF ablation procedure. The disadvantage in current bipolar balloon-based ablation catheters is that the blood flow through the renal artery is blocked while the balloon is inflated increasing the risk to the patient.

Another shortcoming of current renal denervation systems is that even though the physician can observe the positioning of the ablation catheter in the renal artery through contrast media supported X-ray, the physician does not know the location of the sympathetic nerves of the renal artery and therefore does not know the correct and ideal position of the catheter to be placed to make the actual ablation and treatment time as short and efficient as possible. Physicians are essentially performing this procedure blind, with presently available devices and the only available approach to the physician is to perform ablation to all nerves surrounding the renal artery in several places along the renal artery. Even though it is well known that the over-activity of the renal sympathetic nerves are responsible for higher blood pressure in a patient, the actual place or location of the nerve path with the over-activity is not measured to be able to identify the correct location to perform a targeted ablation of the nerve.

Conventional ablation methods and systems have generally been considered satisfactory for their intended purpose. However, there is still a need in the art for improved catheter-based ablation systems. There also remains a need in the art for a system that is easy to make and use. The present disclosure provides a solution for these problems.

### SUMMARY

A catheter assembly can include an elongated catheter having opposed proximal and distal end portions and having an axial lumen extending therethrough, the axial lumen configured to accommodate electrical wires. The assembly can include a distal electrode array configured to have a basket shape in a deployed position and configured to collapse in a collapsed position, the distal electrode array configured to extend from the distal end portion of the elongated catheter in the deployed position and to be selectively contained within the axial lumen or other sheath in the collapsed position.

The assembly can include a plurality of electrical wires electrically connected to the distal electrode array and disposed through the axial lumen, a proximal hub connected to the elongated catheter at the proximal end, and an electrical plug connected to the proximal hub and electrically connected to the plurality of electrical wires. The assembly can include a flexible tube disposed in the axial lumen of the elongated catheter, and a tubing having a side port with a multiway stop cock side port such that the catheter assembly can be flushed. In certain embodiments, the assembly can include an introducer for loading the elongated catheter and/or distal electrode array into a deflectable guiding sheath for delivery of elongated catheter and/or distal electrode array through femoral access into the renal nerves.

The distal electrode array can include two or more (e.g., four) branches configured to be collapsible and adjustable. The branches can be made of pre-shaped shape-memory material wire. In certain embodiments, the shape-memory material wire is covered with a thermoplastic polyurethane (TPU). In certain embodiments, the shape-memory material wires can be terminated with a cylindrical cap.

The distal electrode array can include a plurality of ablation electrodes. For example, a four branch configuration can include sixteen ablation electrodes such that four ablation electrodes can be attached to a top of each of the four branches. Each ablation electrode can be electrically attached to an electrical wire of the plurality of electrical wires. The electrodes can have a thermoplastic polyurethane (TPU) cover with a cutout window to expose the electrodes surface to come in contact with the ablated surface.

The distal electrode array can include two or more orientation electrodes, at least one disposed on two or more of the branches, and configured for orientation under fluoroscopy. In certain embodiments, the orientation electrodes may not be connected to the electrical wires. The axial lumen can be terminated at distal end with adhesive along with a star shaped fixture to hold the plurality of branches in place and allowing the branches to collapse and expand with a guide connected from the distal cap and into the flexible tube.

In certain embodiments, the assembly can include a switching module configured to connect to the electrical plug and the electrodes, and to connect to a recorder module configured to receive signals from the electrode array and an ablation energy generator to provide selection between electrical mapping and ablation energy generation. In certain embodiments, the ablation electrodes and/or the cap and/or the orientation electrodes are made of platinum iridium (e.g., cylindrical platinum iridium electrodes).

In accordance with at least one aspect of this disclosure, an ablation catheter system can include a recorder module configured to receive sympathetic signals from the circumference of the artery to allow the location of the nerve path with the renal sympathetic over-activity, an ablation energy generator configured to output power to the ablation electrodes to cause tissue ablation, and a switching module configured to switch between the recorder module and an ablation energy generator to switch between electrical mapping and ablation of the selected zone with the renal sympathetic over-activity utilizing the same electrode array.

In accordance with at least one aspect of this disclosure, an electrode array for renal ablation can include a basket shape in a deployed position and configured to collapse in a collapsed position, the electrode array configured to extend from a distal end portion of an elongated catheter in the deployed position and to be selectively contained within the axial lumen or other sheath in the collapsed position. The electrode array can include two or more branches configured to be collapsible and adjustable.

The branches can be made of pre-shaped shape-memory material wire. The distal electrode array can include a plurality of ablation electrodes (e.g., each branch having multiple). In certain embodiments, the plurality of electrodes can be sixteen ablation electrodes, with four ablation electrodes attached to a top of each of the branches. Each ablation electrode can be electrically attached to an electrical wire of the plurality of electrical wires. The distal electrode array can include two or more orientation electrodes, at least one disposed on two or more of the branches, and configured for orientation under fluoroscopy. The orientation electrodes may not be connected to the electrical wires. The electrode array can be or include any suitable embodiment of an electrode array disclosed herein, e.g., as described above.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a schematic elevation view of an embodiment of a catheter assembly in accordance with this disclosure;

FIG. 2A is a schematic elevation view of an end effector portion of the catheter assembly as shown in FIG. 1;

FIG. 2B is a schematic elevation view of a portion of an electrode assembly of the embodiment of FIG. 2A;

FIG. 2C is a cross-sectional view of the elongate catheter of the embodiment of FIG. 2A;

FIG. 3 is a schematic diagram of an embodiment of a switching module operatively connected to an electrical mapping module and an RF generator module, and the catheter, e.g., as shown in FIG. 1; and

FIG. 4 is a schematic diagram of the embodiment of FIGS. 1 and 2 being inserted into a renal artery.

### DETAILED DESCRIPTION

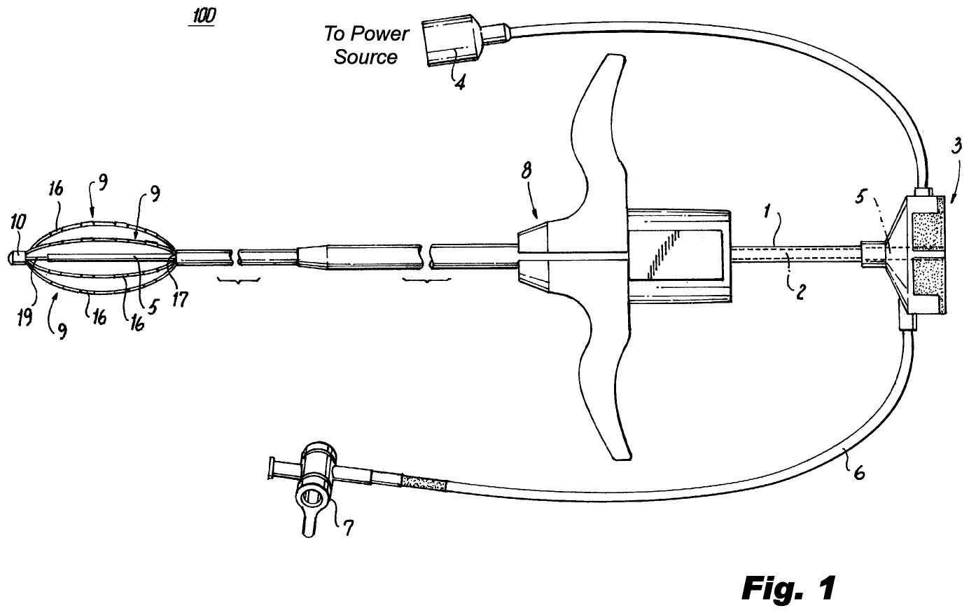

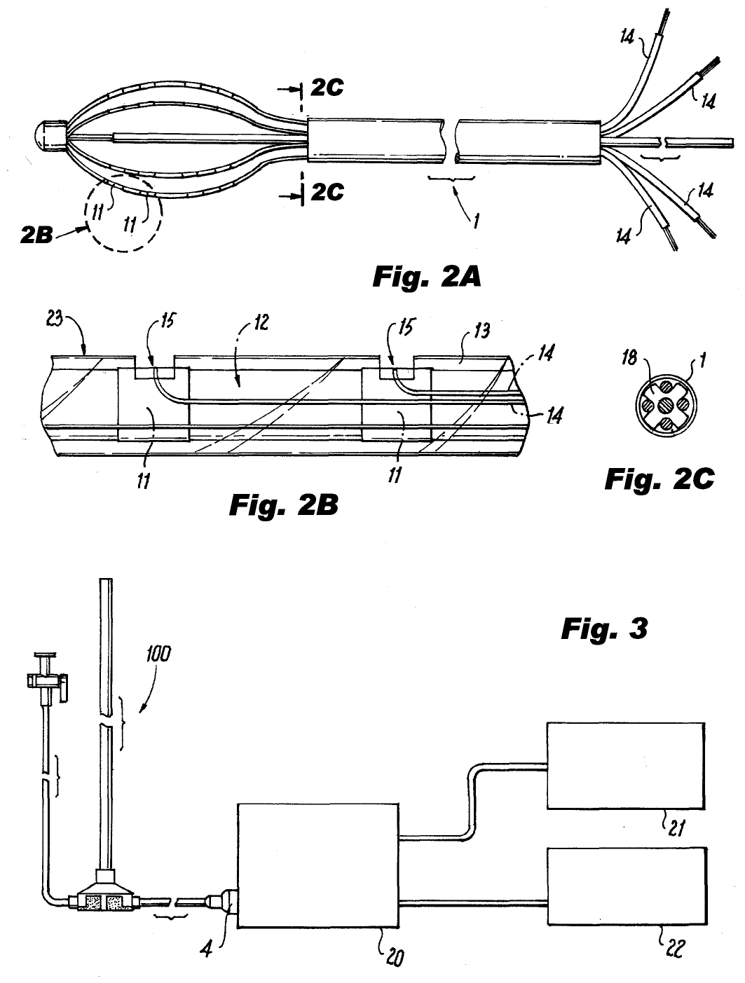

Reference will now be made to the drawings wherein like reference numerals identify similar structural features or aspects of the subject disclosure. For purposes of explanation and illustration, and not limitation, an illustrative view of an embodiment of an ablation catheter in accordance with the disclosure is shown in FIG. 1 and is designated generally by reference character 100. Other embodiments and/or aspects of this disclosure are shown in FIGS. 2A-4. Certain embodiments disclosed herein can be used for ablating the interior walls of the renal artery to reduce renal sympathetic afferent and efferent activity, among other things.

In accordance with at least one aspect of this disclosure, referring to FIGS. 1, 2A, 2B, and 2C, a catheter assembly 100 can include an elongated catheter 1 having opposed proximal and distal end portions and having an axial lumen 2 extending therethrough. The axial lumen 2 can be configured to accommodate electrical wires 14 (e.g., insulated). The assembly 100 can include a distal electrode array 101 configured to have a basket shape (e.g., a skeleton approximating a balloon shape as shown or any other suitable shape) in a deployed position (e.g., as shown) and configured to collapse in a collapsed position (e.g., to be linear to fit within a distal end of the axial lumen 2 or other sheath). The distal electrode array 101 can be configured to extend from the distal end portion of the elongated catheter 1 in the deployed position, e.g., as shown, and to be selectively contained within the axial lumen 2 or other sheath in the collapsed position, for example. Any other suitable relative relationship to the elongated catheter 1 is contemplated herein.

The assembly 100 can include a plurality of electrical wires 14 electrically connected to the distal electrode array 101 and disposed through the axial lumen 2, a proximal hub 3 connected to the elongated catheter 1 at the proximal end, and an electrical plug 4 connected to the proximal hub 3 and electrically connected to the plurality of electrical wires 14. The assembly 100 can include a flexible tube 5 (e.g., made of polyimide or other suitable material) disposed in the axial lumen 2 of the elongated catheter 1. The assembly 100 can include tubing 6 having a side port with a multiway (e.g., two or three way) stop cock side port 7 such that the catheter assembly can be flushed.

In certain embodiments, the assembly 100 can include an introducer 8 for loading the elongated catheter 1 and/or distal electrode array 101 into a deflectable guiding sheath for delivery of elongated catheter 1 and/or distal electrode array 101 through femoral access into the renal nerves. Any suitable introducer 8 is contemplated herein (e.g., an Adelante® Peel-Away Introducer made by Oscor, Inc. Palm Harbor Fl, 34683), e.g., for loading the basket catheter into a Destino™ deflectable guiding sheath made by Oscor, Inc. Palm Harbor Fl, 34683 or any other suitable guiding sheath is contemplated herein.

The distal electrode array 101 can include two or more branches 9 (e.g., four as shown) configured to be collapsible and adjustable. Any other suitable number of branches (e.g., two to six) that form a basket shape is contemplated herein. The branches 9 can extend radially outward, each in a bow shape, e.g., as shown, to for, the basket shape.

The branches 9 can be made of pre-shaped shape-memory (e.g., Nitinol) material wire 12. In certain embodiments, the shape-memory material wire 12 can be covered with a thermoplastic polyurethane (TPU) (e.g., Pellethane®) 13 or any other suitable electrical insulating material. In certain embodiments, the shape-memory material wires 12 can be terminated with a cylindrical cap 10 or any other suitable tip (e.g., a smooth surface).

The distal electrode array 101 can include a plurality of ablation electrodes 11 (e.g., a plurality of electrodes on each branch 9). For example, for a four branch configuration, the distal electrode array 101 can include sixteen ablation electrodes 11, four ablation electrodes 11 attached (e.g., glued) to a top of each of the four branches 9, e.g., as shown. Any other suitable number of electrodes 11 is contemplated herein.

Each ablation electrode 11 can be electrically attached (e.g., laser welded) to an electrical wire 14 (e.g., insulated wires 15) of the plurality of electrical wires 14. Each pair of electrodes 11 can have a thermoplastic polyurethane (TPU) cover therebetween. In certain embodiments, each electrode 11 can have a TPU cover 23 with a cutout window to expose the electrodes surface to come in contact with the ablated surface, e.g., as shown in FIG. 2C. As shown, a first pair of electrodes 11 on each branch can be positioned distally of an apex of the curvature of each branch 9, and a second pair of electrodes 11 can be placed proximally of the apex of each branch 9. Any other suitable arrangement is contemplated herein. Any other suitable material other than TPU is contemplated herein.

The distal electrode array 101 can include two or more orientation electrodes 16, at least one disposed on two or more of the branches 9, e.g., as shown, and configured for orientation under fluoroscopy for example. The orientation electrodes 16 may not be connected to the electrical wires 14. The orientation electrodes 16 can be placed distally of the ablation electrodes 11 on each branch 9. Any other suitable position is contemplated herein.

The axial lumen 2 can be terminated at distal end with adhesive 17 along with a star shaped fixture 18 to hold the plurality of branches 9 in place and allowing the branches 9 to collapse and expand with a guide 19 connected from the distal cap 10 and into the flexible tube 5. The guide 19 can be made of any suitable rigid or semi-rigid material (e.g., stainless steel), an can be attached to flexible tube 5 (e.g., bonded) in any suitable manner to allow the guide 19 to be pulled by the flexible tube 5 (e.g., to move to the deployed position). Any other suitable structure configured to allow the distal electrode array 101 to move between the deployed position and the collapsed position is contemplated herein.

In certain embodiments, referring to FIG. 3 the assembly 100 can include a switching module 20 configured to connect to the electrical plug 4 and the electrodes 11, 16, and to connect to a recorder module 21 configured to receive signals from the electrode array 101 and an ablation energy generator 22 (e.g., an RF generator) to provide selection between electrical mapping and ablation energy generation. The switching module 20 can include any suitable hardware and/or software module(s) configured to allow switching between mapping and ablation (e.g., due to a manual input to switch, or in accordance with any suitable schedule or logic).

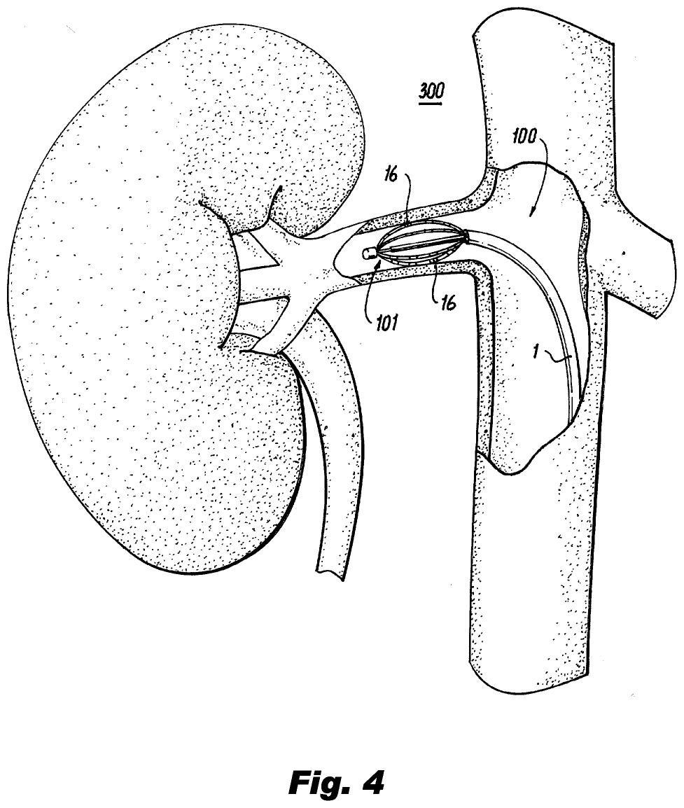

FIG. 4 shows a schematic diagram of the embodiment of FIGS. 1 and 2 being inserted into a renal artery. As shown, the elongate catheter 1 can be compliant and/or steerable (e.g., configured to bend or make angled turns (e.g., 90 degrees). Any suitable arrangement for steering the device into a suitable anatomical location is contemplated herein.

In certain embodiments, the ablation electrodes and/or the cap and/or the orientation electrodes are made of platinum iridium (e.g., cylindrical platinum iridium electrodes). Any other suitable material and/or shape is contemplated herein.

In accordance with at least one aspect of this disclosure, an ablation catheter system (e.g., system 300 as shown in FIG. 3) can include a recorder module 21 configured to receive sympathetic signals from the circumference of the artery to allow the location of the nerve path with the renal sympathetic over-activity. The system 300 can include an ablation energy generator 22 configured to output power to the ablation electrodes 11 to cause tissue ablation. The system 300 can include a switching module 20 configured to switch between the recorder module 21 and an ablation energy generator 22 to switch between electrical mapping and ablation of the selected zone with the renal sympathetic over-activity utilizing the same electrode array 101.

In accordance with at least one aspect of this disclosure, an electrode array 101 for renal ablation can include a basket shape in a deployed position and configured to collapse in a collapsed position, the electrode array 101 configured to extend from a distal end portion of an elongated catheter in the deployed position and to be selectively contained within the axial lumen or other sheath in the collapsed position. The electrode array 101 can include two or more (e.g., four) branches 9 configured to be collapsible and adjustable.

The branches 9 can be made of pre-shaped shape-memory material wire. The distal electrode array 101 can include a plurality of electrodes on each branch. For example, certain embodiments can include sixteen ablation electrodes with four ablation electrodes attached to a top of each of the branches 9. Each ablation electrode can be electrically attached to an electrical wire 14 of the plurality of electrical wires 14. The distal electrode array 101 can include two or more orientation electrodes 16, at least one disposed on two or more of the branches 9, and configured for orientation under fluoroscopy. The orientation electrodes 16 may not be connected to the electrical wires 14 that are connected to the ablation electrodes 11, for example. The electrode array 101 can be or include any suitable embodiment of an electrode array 101 disclosed herein, e.g., as described above.

In accordance with at least one aspect of this disclosure, certain embodiments of a system (e.g., system 100) for use in a renal denervation procedure can include a catheter having proximal and distal end portions, a series of splines with pre-shaped geometry with memory to collapse and return to a basket shape, the electrodes array assembled over the basket splines to measure the renal sympathetic activity around the renal artery and the same electrodes array on the distal end portion of the catheter for delivering energy to ablate the renal tissue with nerves surrounding the renal artery. Each pair of electrodes can have a Pellethane® cover between them to reduce current density at the edges which protects the artery wall from ablation and channels the current flow through the tissue beyond walls for an effective ablation of the target area.

The system can further include a catheter handle (e.g., proximal hub 3) at the proximal end portion of the catheter wherein the handle is connectable to a multiplexer or switch box that is configured to either perform mapping of renal sympathetic nerve activity or provide energy to a selectable pair of electrodes for ablation of the renal artery. The catheter handle can include an actuation portion for facilitating bidirectional steering of the distal end portion of the catheter within the renal artery. An overall diameter of the catheter can be less than about 6 F, for example. Any suitable handle structure, e.g., as appreciated by those having ordinary skill in the art, for steering the catheter or size is contemplated herein.

The distal end portion of the catheter can have a generally basket shaped configuration. In some embodiments, the distal end portion of the catheter can have two to six splines forming the basket shape, for example. Any suitable number of splines (e.g., branches 9 as described above) is contemplated herein. The system can further include a radio frequency generator operatively connected to the catheter handle to provide energy to the plurality of electrodes for ablation of the renal artery.

In at least one aspect of this disclosure, a method can include inserting the catheter into a renal artery and sensing a condition associated with a nerve of a renal artery using the electrodes on the catheter. The method can further include determining whether to ablate tissue based the sensed condition of the nerves. The method can further include ablating tissue if the nerves are sensed to be over-active. The method can include any other suitable method(s) and/or portion(s) thereof.

In at least one aspect of this disclosure, a catheter can include a catheter body defining a distal end portion and a proximal end portion, and an electrode array 101 for sensing a renal sympathetic nerve, the electrodes disposed on the distal end portion of the catheter body. The electrodes can be configured to sense the electro-chemical signals from the renal sympathetic nerves. The catheter can further include the electrode array 101 to be electrically connected to an electro surgical energy source generator. In certain embodiments, a diameter of the catheter body can be less than about 6 F.

The catheter can further include a catheter handle at a proximal end portion of the catheter body wherein the handle is connectable to a generator that is configured to provide energy to the any selectable pair of electrodes for ablation of a renal artery location. The catheter handle can include an actuation portion for steering the distal end portion of the catheter body within the renal artery. Any suitable handle and/or steering assembly is contemplated herein.

Embodiments can include a system for use in a renal denervation procedure having a catheter having proximal and distal end portions, a sensor configuration to monitor the condition of the nerves surrounding the renal artery, the sensor array operatively associated with the distal end portion of the catheter, and the same sensor array on the distal end portion of the catheter for delivering energy to renal surrounding tissue. A catheter can include a catheter body defining a distal end portion and a proximal end portion, and a sensor array for sensing a renal sympathetic nerve activity, the same sensor array serves dual purpose on the distal end portion of the catheter body. The sensor array can be configured to sense the electrical signals from the renal sympathetic nerves surrounding the renal artery and selectively perform renal ablation at the target area.

As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A catheter assembly comprising:
an elongated catheter having opposed proximal and distal end portions and having an axial lumen extending therethrough, the axial lumen configured to accommodate electrical wires; and
a distal electrode array configured to have a basket shape in a deployed position and configured to collapse in a collapsed position, the distal electrode array configured to extend from the distal end portion of the elongated catheter in the deployed position and to be selectively contained within the axial lumen or other sheath in the collapsed position.

2. The assembly of claim 1, further comprising:
a plurality of electrical wires electrically connected to the distal electrode array and disposed through the axial lumen;
a proximal hub connected to the elongated catheter at the proximal end; and
an electrical plug connected to the proximal hub and electrically connected to the plurality of electrical wires.

3. The assembly of claim 2, further comprising:
a flexible tube disposed in the axial lumen of the elongated catheter, and
a tubing having a side port with a multiway stop cock side port such that the catheter assembly can be flushed.

4. The assembly of claim 3, further comprising an introducer for loading the elongated catheter and/or distal electrode array into a deflectable guiding sheath for delivery of elongated catheter and/or distal electrode array through femoral access into the renal nerves.

5. The assembly of claim 1, wherein the distal electrode array includes two or more branches configured to be collapsible and adjustable.

6. The assembly of claim 5, wherein the two or more branches are made of pre-shaped shape-memory material wire.

7. The assembly of claim 6, wherein the shape-memory material wire is covered with a thermoplastic polyurethane (TPU).

8. The assembly of claim 6, wherein the shape-memory material wires are terminated with a cylindrical cap.

9. The assembly of claims 2 and 5, wherein the two or more branches include four branches, wherein the distal electrode array include sixteen ablation electrodes, four ablation electrodes attached to a top of each of the four branches, wherein each ablation electrode is electrically attached to an electrical wire of the plurality of electrical wires.

10. The assembly of claim 9, wherein each electrode can have a thermoplastic polyurethane (TPU) cover with a cutout window to expose the electrodes surface to come in contact with the ablated surface.

11. The assembly of claim 10, wherein the distal electrode array includes two or more orientation electrodes, at least one disposed on two or more of the branches, and configured for orientation under fluoroscopy, wherein the orientation electrodes are not connected to the electrical wires.

12. The assembly of claim 11, wherein the axial lumen is terminated at distal end with adhesive along with a star shaped fixture to hold the four branches in place and allowing the branches to collapse and expand with a guide connected from the distal cap and into the flexible tube.

13. The assembly of claim 11, wherein further comprising a switching module configured to connect to the electrical plug and the electrodes, and to connect to a recorder module configured to receive signals from the electrode array and an ablation energy generator to provide selection between electrical mapping and ablation energy generation.

14. The assembly any of the preceding claims wherein the ablation electrodes and/or the cap and/or the orientation electrodes are made of platinum iridium.

15. An ablation catheter system, comprising:
a recorder module configured to receive sympathetic signals from the circumference of the artery to allow the location of the nerve path with the renal sympathetic over-activity;
an ablation energy generator configured to output power to the ablation electrodes to cause tissue ablation;
a switching module configured to switch between the recorder module and the ablation energy generator to switch between electrical mapping and ablation of a selected zone with the renal sympathetic over-activity utilizing the same electrode array.

16. An electrode array for renal ablation comprising a basket shape in a deployed position and configured to collapse in a collapsed position, the electrode array configured to extend from a distal end portion of an elongated catheter in the deployed position and to be selectively contained within the axial lumen or other sheath in the collapsed position.

17. The electrode array of claim 16, wherein the electrode array includes four branches configured to be collapsible and adjustable.

18. The electrode array of claim 17, wherein the four branches are made of pre-shaped shape-memory material wire.

19. The electrode array of claim 18, wherein the distal electrode array include sixteen ablation electrodes, four ablation electrodes attached to a top of each of the four branches, wherein each ablation electrode is electrically attached to an electrical wire of the plurality of electrical wires, wherein the distal electrode array includes two or more orientation electrodes, at least one disposed on two or more of the branches, and configured for orientation under fluoroscopy, wherein the orientation electrodes are not connected to the electrical wires.
